(19) United States
(12) Patent Application Publication
(10) Pub. No.: US 2010/0291224 A1
(43) Pub. Date: Nov. 18, 2010

(54) NANOSTRUCTURES, METHODS OF PREPARING AND USES THEREOF

(76) Inventors: Yen Wah Tong, Singapores (SG); Chau Jin Tan, Singapore (SG); Hong Gap Chua, Singapore (SG); Kwee Hong Ker, Singapore (SG); Niranjani Sankarakumar, Singapore (SG)

Correspondence Address: KNOBBE MARTENS OLSON & BEAR LLP, 2040 MAIN STREET, FOURTEENTH FLOOR, IRVINE, CA, 92614, US

(21) Appl. No.: 12/811,690

(22) PCT Filed: Jan. 5, 2009
(86) PCT No.: PCT/SG09/00002

§ 371 Date: Jul. 2, 2010

Related U.S. Application Data
(60) Provisional application No. 61/018,742, filed on Jan. 3, 2008.

(51) Int. Cl. A61K 9/16; G01N 33/53; G01N 33/538; G01N 33/545; B05D 7/00; C12Q 1/70

(52) U.S. Cl. 424/496; 427/222; 427/127; 427/2.1; 422/101; 435/5; 977/773; 977/904

(57) ABSTRACT

The present invention provides a core-shell nanostructure comprising:
a hydrophobic polymeric core; and
a hydrophobic polymeric shell on the core,
wherein the shell comprises at least one binding site for binding at least one target agent. In particular, the nanostructure has a red-blood cell morphology.

The present invention also provides a method for preparing the nanostructure and uses of the nanostructure.

### FIELD OF THE INVENTION

The present invention relates to nanostructures and methods of preparing the nanostructures. The present invention also provides uses of the nanostructures. In particular, the present invention relates to the field of separation (industrial purification of proteins), analytical chemistry, therapeutics, biosensing and/or bioimaging.

### BACKGROUND OF THE ART

Molecular imprinting has been widely recognized as the most feasible approach to prepare synthetic receptors and/or antibodies for predetermined template molecules by imparting a predetermined molecular recognition property onto synthetic materials such as polymers. Interest in molecular imprinting has been widely increasing in view of potential wide applications of molecularly imprinted polymers (MIPs) in the fields of separation, catalysis, analytical chemistry, and biosensing. Compared to its biological counterparts, enzymes and antibodies, MIPs can not only display comparable molecular selectivity, they are also more robust, reusable, and most of all, easy and inexpensive to prepare. Therefore, MIPs represent a new class of materials that could mimic and possibly replace their biological equivalents.

To date, most commercially available MIPs are synthesised using a conventional bulk polymerization approach. In the methodology of the conventional bulk polymerization, functional monomers are first allowed to interact and complex with template molecules in a homogeneous pre-polymerization solution. Then, in the presence of excess cross-linking monomers, the polymerization reaction is initiated and upon completion of the polymerization reaction, a large imprinted polymer bulk is obtained. As post-treatment, the polymer bulk is ground and sieved to obtain the particles of desired sizes.

Despite the ease and simplicity of the approach, the MIPs prepared using the conventional bulk polymerization method are often sharp and irregular thus restricting the application of these MIPs in areas such as liquid chromatography. In addition, although conventional bulk polymerization has been applied successfully to imprint small molecules like peptides and drugs, success associated with macromolecules such as proteins has been limited. One of the major difficulties faced by these large molecules for the imprinting application lies in diffusion limitations, due to the bulkiness of the protein molecules which restricts their diffusion into and out of binding sites found beneath the surface of the MIPs. Furthermore, with poor thermal dispersion, conventional bulk imprinting is not suitable to be employed in an industrial scale.

MIPs with a core-shell nanostructure may be made according to the method described in Perez et al (2000) and Perez-Moral N., Mayes A. G., (2004). However, the core-shell MIPs synthesised according to Perez et al. have binding sites located within the shell of the core-shell MIPs thus making diffusion of the template to the binding site difficult and removal of the template post-polymerisation inefficient.

Accordingly, there is a need in this field of technique of improved nanostructures and methods of preparing the nanostructures.

### SUMMARY OF THE INVENTION

The present invention addresses the problems above, and in particular provides a novel method of preparing nanostructure(s). The nanostructures prepared from the method of the present invention may be used for applications such as in imaging, detection of target agent(s) and/or method of treatment.

According to a first aspect of the present invention, there is provided a core-shell nanostructure comprising a hydrophobic polymeric core and a hydrophobic polymeric shell on the core, wherein the shell comprises at least one binding site on for binding at least one target agent.

The core-shell nanostructure may have a red-blood cell morphology. In particular, the red-blood cell morphology may provide the nanostructure with a large surface area to volume ratio.

The binding sites may be on the outer face of the shell. In particular, the binding sites may be substantially on the outer face of the shell.

The core may be magnetic. In particular, the core may comprise at least one magnetic material.

The binding site may be formed from removal of at least one template with conformation of the target agent and wherein the template, prior to removal, may be connected to the core by a linker. In particular, with the removal of the template, the linker may remain within the shell thus being able to link the core to the target agent. More in particular, the conformation of the target agent is complementary to the conformation of the binding site.

In particular, the target agent may at least be one small molecule. In particular, the small molecule may be at least one hydrophilic drug, hydrophobic drug, vitamin, polysaccharide, and/or steroid. The steroid may be a sterol, for example, cholesterol. In particular, the cholesterol may be in a complex with at least one protein to form a lipoprotein. More in particular, the lipoprotein may be a high density lipoprotein (HDL), low density lipoprotein (LDL) and/or very low density lipoprotein (VLDL).

The target agent may be a large molecule, for example, protein, DNA, virus, carbohydrate, macrocycle and/or a cell comprising at least one portion of stable conformation. In particular, the cell comprising the stable conformation comprises a static structure.

In particular, the target agent may at least be one virus. The virus may be selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like. The target agent may be a full or a part of the virus. In particular, the part of the virus may be a protein coat, lipid or polysaccharide envelope or the like of the virus.

The core-shell nanostructure of the present invention may further comprise at least one label which is detectable when the core-shell nanostructure may be bound to the target agent. In particular, the label may be a reporter molecule that may be activated when the target agent binds to the nanostructure.

According to another aspect, the present invention provides a method of preparing at least one core-shell nanostructure comprising at least one binding site, for binding at least one target agent, the method comprising:
(a) providing at least one first hydrophobic polymer to form a core;
(b) providing at least one template to bind to the core, wherein the template comprises the conformation of at least one target agent;
(c) providing at least one second hydrophobic polymer to form a shell on the core and on a portion of the template; and
(d) removing the template, to form at least one core-shell nanostructure comprising at least one binding site, for binding the target agent.

The core-shell nanostructure may have a red-blood cell morphology.

The binding sites, in particular when the template is a large molecule, may be on the outer face of the shell. In particular, the binding sites may be substantially on the outer face of the shell.

The core may be magnetic. In particular, the core may comprise at least one magnetic material.

The first and/or second hydrophobic polymer may be any polymer that may be hydrophobic. In particular, the hydrophobic polymer may be selected from the group consisting of vinyl acrylate polymers, vinyl acetate polymers, acrylamides, nitrile polymers and/or a mixture thereof. More in particular, the hydrophobic polymer may be poly(methyl methacrylate), poly(ethylene glycol di methacrylate), poly(methyl acrylate), poly(hydroxylethyl methacrylate), poly(vinyl acetate), poly(vinyl alcohol), poly(vinyl acrylamide), and other chain polymers that have hydrophobic functional groups such as alcohol, carboxyl, amide/amine and the like.

The target agent may be ≦1 μm in size. In particular, the target agent may be at least one small molecule. In particular, the small molecule may be at least one hydrophilic drug, hydrophobic drug, vitamin, polysaccharide, and/or steroid. More in particular, the steroid may be cholesterol. The cholesterol may be in a complex with at least one protein to form a lipoprotein. In particular, the lipoprotein may be a high density lipoprotein (HDL), low density lipoprotein (LDL) and/or very low density lipoprotein (VLDL).

The target agent may be a protein, DNA, virus, carbohydrate, macrocycle and/or a cell comprising at least one portion of stable conformation. In particular, the cell comprising the stable conformation comprises a static structure.

The target agent may be directly bound to the core or the target agent may be bound via a linker to the core in step (b) above according to any method of the present invention. In particular, the target agent may be bound to the core by covalent bonding in step (b) of the method of the present invention.

The step of removing the target agent in step (d) of the method of the present invention may be carried out by hydrolysis. In particular, the hydrolysis may be alkaline or acid hydrolysis.

In another aspect of the present invention, there is provided at least one core-shell nanostructure obtainable or obtained according to the method of the present invention.

In another aspect of the present invention, there is provided a nanostructure for binding at least one virus, the nanostructure comprising at least one hydrophobic polymer, and at least one binding site on the outer face of the nanostructure for binding the virus.

The virus may be selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like. The target agent may be a full or a part of the virus. In particular, the part of the virus may be a protein coat, lipid or polysaccharide envelope and the like of the virus. In particular, the conformation of the virus and/or part thereof may be complementary to the conformation of the binding site.

According to another aspect, the nanostructure of the present invention may be for use as antibody substitute. In particular, the antibody substitute may be synthetic. The nanostructure of the invention may also be used as an enzyme substitute by having an enzyme-like activity. Accordingly, there are also provided test and/or kits using the nanostructure of the invention as antibody or fragment thereof substitute and/or enzyme substitute.

According to a further aspect, the present invention provides a method of imaging of at least one subject, the method comprising:
(a) administering the nanostructure according to any aspect of the present invention to at least one subject;
(b) allowing the nanostructure to contact the target agent to form at least one nanostructure-target agent complex; and
(c) detecting the presence of the nanostructure-target agent complex in the subject.

According to another aspect, the present invention provides a method of detecting and/or imaging at least one target agent in at least one biological sample, the method comprising:
(a) collecting at least one biological sample from a subject;
(b) contacting the nanostructure according to any aspect of the present invention to the biological sample;
(c) allowing the nanostructure to contact the target agent to form at least one nanostructure-target agent complex; and
(d) detecting the presence of the nanostructure-target agent complex in the biological sample of the subject.

Any biological sample obtained from a subject may be used for the purpose of the present invention. For example, the biological sample may be blood, serum, spinal fluid, saliva and/or urine. The core-shell nanostructure may further comprise at least one label which is detectable when the core-shell nanostructure may be bound to the target agent. In particular, the label may be a reporter molecule that may be activated when the target agent binds to the nanostructure.

According to one aspect the present invention provides a method of detecting at least one target agent and/or of diagnosis of at least one disorder, the method comprising:
(a) collecting at least one biological sample from a subject;
(b) administering the nanostructure according to any aspect of the present invention to the biological sample;
(c) allowing nanostructure to contact the target agent to form at least one nanostructure-target agent complex; and
(d) detecting the presence of the nanostructure-target agent complex in the biological sample;
   wherein detection of the nanostructure-target agent complex indicates the presence of the target agent and/or disorder in the subject.

Accordingly, the invention may be used to detect the presence of a drug, virus, or the like in a subject or a sample from a subject. Accordingly, there is also provided and assay and/or a kit comprising the nanostructure according to the invention for use in diagnostic test. Any biological sample obtained from a subject may be used for the purpose of the present invention. For example, the biological sample may be blood, serum, spinal fluid, saliva and/or urine. In particular, the disorder may be at least one viral infection. More in particular, the viral infection may be a result of the virus selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like.

According to another aspect of the present invention there is provided a method for selective binding, separation, and/or purification of at least one target agent from a mixture of agents, wherein the mixture of agents comprises the target agent and at least one non-target agent and wherein the method comprises:
(a) contacting the nanostructure according to any aspect of the present invention to a mixture of agents;
(b) allowing the binding of the nanostructure to the target agent in the mixture of agents to form at least one nanostructure-target agent complex;
(c) separating the nanostructure-target agent complex from the mixture of agents; and
(d) separating the target agent from the nanostructure-target agent complex to obtain the target agent.

According to one aspect of the present invention there is provided, a method of treatment of at least one disorder in a subject, the method comprising, administering the nanostructure according to any aspect of the present invention to the subject with the disorder.

The disorder may be at least one viral infection. In particular, the viral infection may be a result of the virus selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like.

According to one aspect, the present invention provides a use of the nanostructure according to any aspect of the present invention for the preparation of a medicament for the treatment of at least one disorder. The disorder may be at least one viral infection. In particular, the viral infection may be a result of the virus selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like. In particular, the nanostructure according to the invention may be useful as delayed-releasing of an agent, like a drug in the body of a subject. The drug may be a drug for the treatment of cancer.

According to another aspect, the present invention provides a nanostructure according to any aspect of the present invention for use in the treatment of a disorder. The disorder may be at least one viral infection. In particular, the viral infection may be a result of the virus selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like.

According to another aspect, the present invention provides a pharmaceutical composition comprising the nanostructure according to any aspect of the present invention. The pharmaceutical composition may further comprise at least one pharmaceutically acceptable excipient, diluent, carrier and/or adjuvant.

### BRIEF DESCRIPTION OF THE FIGURES

FIG. 1 shows a schematic representation of the imprinting strategy to form at least one core-shell nanostructure comprising at least one binding site on the outer face of the shell for binding at least one target agent. The imprinting strategy is based on surface immobilization of template, in this case Bovine Serum Albumin (BSA) molecule, with a series of surface modification steps of the support beads prior to polymerization using a two-stage core-shell miniemulsion polymerization.

FIG. 2 shows a picture of the set-up of the miniemulsion polymerization system at the polymerisation stage.

FIG. 3 shows microscopic observation of the prepared particles. Field-Emission Scanning Electron Microscope (FESEM) images of (a) support particles, (b) imprinted particles based on immobilized template molecules (iMIP), (c) nonimprinted particles (iNIP), and (d) TEM images illustrating the successful encapsulation of the Fe₃O₄ magnetite.

FIG. 4 shows the results of (a) BSA batch rebinding tests, +, p<0.05; −, p<0.08; (b) Lysozyme (Lys) batch rebinding tests in water (white blocks represent nonimprinted particles with similar surface functionalization (iNIP); black blocks represent imprinted particles based on immobilized template molecules (iMIP); crosshatch blocks represent nonimprinted particles without the surface modification (fNIP); and broken crosshatch blocks represent imprinted particles with non-immobilized (or free) template molecules (fMIP).

FIG. 5 shows the results of a competitive rebinding test using the MIP particles obtained using the method illustrated in FIG. 1 at the initial concentration of 1.8 mg/ml where p<0.01. An excellent template recognition property was displayed by the MIP particles where a maximum loading of −890 nmol BSA/g polymer, 7 times greater than that of the NIP was shown. The MIP displayed significantly higher BSA loading than Lysozyme (Lys) loading as BSA was the templated protein and Lys the non-templated protein. (The black represents BSA and the white Lys. * is used to show that no significant adsorption was observed).

FIG. 6 shows the rebinding kinetic behaviour of the nanostructures (black squares represent iNIP; black circles represent, iMIP; black upright triangles represent, fNIP; and black inverted triangles represent, fMIP) in water.

FIG. 7 shows an illustration of virus surface imprinting method through mini-emulsion polymerization.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

### DEFINITIONS

The term “nanostructure” as used herein refers to an extremely small particle. The nanostructure prepared from the method according to any aspect of the present invention may comprise at least one dimension having size ≦1000 nm. For example, ≦700 nm, 500 nm, 300 nm or 100 nm, in particular, ≦50 nm and even more in particular, less than 50 nm. More in particular, the nanostructure may comprise at least one dimension of size ≦25 nm, and even more in particular the nanostructure may comprise at least one dimension of size ≦10 nm or ≦5 nm. The dimension may refer to the average diameter of the nanostructure. The term “nanostructure” may be used interchangeably with “nanoparticle”.

The term “hydrophobic polymer” is used herein to mean any polymer resistant to wetting, or not readily wet, by water, i.e., having a lack of affinity for water. Non-limiting examples of hydrophobic polymers include, by way of illustration only, comprising one or more or a mixture thereof of the following: polyolefins, such as polyethylene, poly(isobutene), poly(isoprene), poly(4-methyl-1-pentene), polypropylene, ethylene-propylene copolymers, ethylene-propylene-hexadiene copolymers, and ethylene-vinyl acetate copolymers; metallocene polyolefins, such as ethylene-butene copolymers and ethylene-octene copolymers; styrene polymers, such as poly(styrene), poly(2-methylstyrene), and styrene-acrylonitrile copolymers having less than about 20 mole-percent acrylonitrile; vinyl polymers, such as poly(vinyl butyrate), poly(vinyl decanoate), poly(vinyl dodecanoate), poly(vinyl hexadecanoate), poly(vinyl hexanoate), poly(vinyl octanoate), and poly(methacrylonitrile); acrylic polymers, such as poly(n-butyl acetate), and poly(ethyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(do-decyl methacrylate), poly(ethyl methacrylate), poly(2-ethylhexyl methacrylate), poly(n-hexyl methacrylate), poly(phenyl methacrylate), poly(n-propyl methacrylate), and poly(octadecyl methacrylate); polyesters, such a poly(ethylene terephthalate) and poly(butylene terephthalate); and polyalkenes and polyalkynes, such as polybutylene and polyacetylene.

The term “polyolefin” is used herein to mean a polymer prepared by the addition polymerization of one or more unsaturated monomers which contain only carbon and hydrogen atoms. Examples of such polyolefins include but are not limited to polyethylene, polypropylene, poly(1-butene), poly(2-butene), poly(1-pentene), poly(2-pentene), poly(3-methyl-1-pentene), poly(4-methyl-1-pentene), and the like. In addition, such term is meant to include blends of two or more polyolefins and random and block copolymers prepared from two or more different unsaturated monomers. Because of their commercial importance, the most desired polyolefins are polyethylene and polypropylene.

The hydrophobic polymer also may contain minor amounts of additives as is customary in the art. For example, the hydrophobic polymer may contain pigments, delustrants, antioxidants, antistatic agents, stabilizers, oxygen scavengers, and the like.

The term “binding site” as used herein refers to a region of the nanostructure of the present invention for interacting with a target agent. In particular, the conformation of the binding site may be complimentary to the conformation of the target agent. The binding site may totally or partially be in contact with the target agent. The binding agent may be on the outer face of the shell of the nanostructure. In particular, in case of large molecule, the binding agent may be on the outer face or substantially on the outer face of the shell of the nanostructure. If the target agent is a small molecule, the binding site may be substantially on the outer face of the nanostructure and, in part, also in the inner part of the nanostructure, within the shell of the nanostructure.

The success or failure of obtaining a desirable molecular recognition function depends on how precisely the selective cavity (target-molecule recognition field) is constructed in the polymer. This is greatly influenced by the design of interaction patterns between the target agent and the functional monomer. The patterns of interactions can be broadly classified into a non-covalent bonding type and a covalent bonding type. In the former, a target agent and a functional monomer are complexed in a pre-polymerization mixture by non-covalent bonding such as hydrogen bonding or electrostatic interactions. In the latter, the complex is synthesized and isolated prior to polymerization with a cross-linker. These methods are selected according to the chemical properties of the target molecule, so that the optimum effect can be obtained. In the molecular imprinting method, the construction of the binding site by the functional monomer and the crosslinking monomer proceeds from the target agent. This optimizes the binding site in terms of entropy, and enables the molecular recognition field to be tailored.

The term “target agent” as used herein refers to any agent which has one or more functional groups and may have a stable and/or static conformation. In particular, the target agent may be any agent that may be ≦1 μm in size. In particular the target agent may at least be one hydrophilic drug, hydrophobic drug, vitamin, protein, polysaccharide, virus, DNA, carbohydrate, macrocycle, steroid and/or at least one portion of a cell comprising at least one stable conformation. In particular, the target agent may be amphiphillic such that it may be partially in contact with the binding site and thus partially encapsulated by the shell of the nanostructure. An example of a cell comprising at least one portion of stable conformation may be a parasite, for instance, a parasitic protozoan. A non-limiting example is the Plasmodium species which is responsible for malaria. The target agent should not be polymerisable with the polymer used for the preparation of the core and/or shell. For the purposes of the present invention, the target agent may also be distinguished in “small molecule” and “large molecule”.

The term “small molecule” as used herein refers to a small organic compound that is biologically active but is not a polymer. In particular, small molecules are molecules with molecular mass (m) (or molecular weight (MW)) less than 2000. A small molecule may or may not include monomers or primary metabolites, in fact it may be generally used to denote molecules that are not protein which play an endogenous or exogenous biological role, such as cell signalling, are used as a tool in molecular biology or are a drug in medicine. Small molecules may be compounds that may be natural (such as secondary metabolites) or artificial (such as antiviral drugs). Examples of small molecules may be hydrophilic or hydrophobic drugs, vitamins, polysaccharides and/or steroids. Other molecules known to a skilled person may also be within the definition of “small molecule”.

“Large molecule” or “macromolecule” as used herein refers to molecules like proteins, viruses, DNA, carbohydrates, macrocycles and/or a cell with at least one portion of stable conformation. A macrocycle is, as defined by IUPAC, “a cyclic macromolecule or a macromolecular cyclic portion of a molecule”.

The term “cell” as used herein refers to a structural and functional unit of all known living organisms containing nuclear and cytoplasmic material enclosed by a semi permeable membrane. In particular, the cell used herein as a target agent may have a stable and static conformation.

The term “label” as used herein refers to an agent capable of detection, for example by ELISA, spectrophotometry, flow cytometry, or microscopy. For example, a label may be attached to a nanostructure or to a target agent, thereby permitting detection of the target agent. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

The term “subject” as used herein refers to living multi-cellular vertebrate organisms, a category that includes human and non-human mammals.

The term “superparamagnetism” as used herein refers to a form of magnetism. A superparamagnetic material may compose of small ferromagnetic clusters (e.g. crystallites), but where the clusters are so small that they can randomly flip direction under thermal fluctuations. As a result, the material as a whole may not be magnetized except in an externally applied magnetic field.

The term “static” as used herein refers to a state pertaining to or characterized by a fixed or stationary condition showing little or no change and lacking movement, development, or vitality. In particular, the target agent used herein may have a static conformation.

The term “stable” as used herein refers to a state that may be able or likely to continue or last which may be firmly established, enduring or permanent. In particular, a stable state may be resistant to molecular or chemical change. More in particular, a portion of a cell with a stable conformation is one that has a fixed structure that may be recognised by a binding site of the nanostructure of the present invention.

The term “conformation” as used herein refers to an atomic spatial arrangement that results from rotation of carbon atoms about single bonds within an organic molecule. In particular, the conformation of a binding site refers to the physical state of the binding site.

The term “portion” as used herein refers to a part of any whole, either separated from or integrated with it. In particular, a portion of the template refers to a part that is in contact with the binding site whilst the remaining part is not in contact with the binding site.

The term “outer face” as used herein refers to the, outside, exterior boundary or uppermost layer of the nanostructure. In particular, the outer face of the nanostructure refers to the surface of the shell of the nanostructure that is free to be exposed to surrounding and/or to bind to at least one target agent.

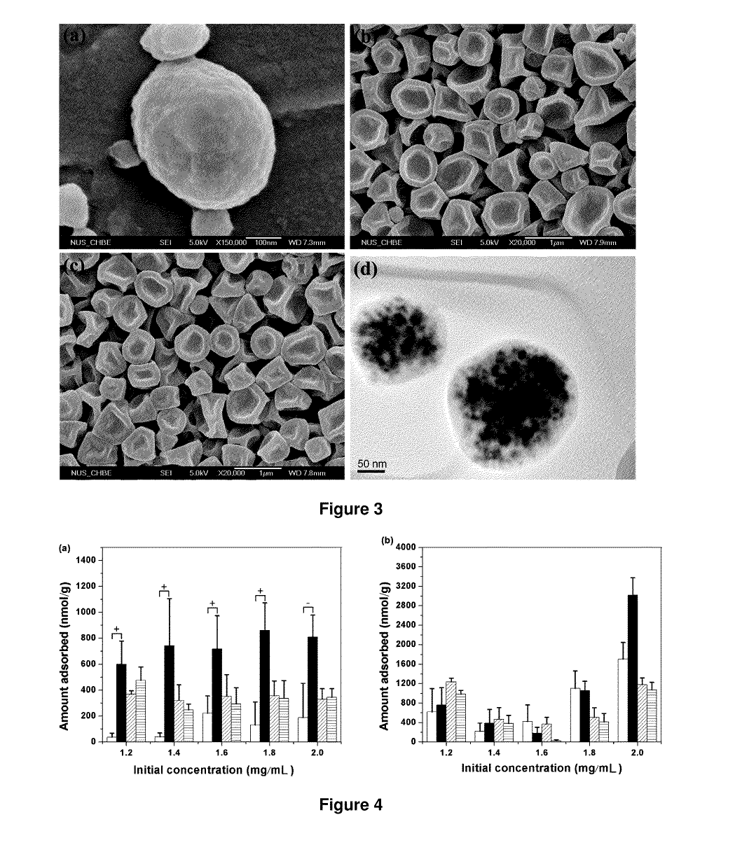

The term “red blood cell morphology” refers to the form and/or structure of the red blood cell (as shown in FIG. 3). In particular, the red blood cell may be flexible and malleable and has a large surface area to volume ratio enabling to be an efficient means of transport.

Description

In view of the limitations posed by the conventional approach for protein imprinting, the present invention provides novel protein surface-imprinted submicron polymeric particles based on template immobilization through a 2-stage core-shell miniemulsion polymerization. The core-shell nanostructure may comprise a hydrophobic polymeric core and a hydrophobic polymeric shell on the core, wherein the shell comprises at least one binding site for binding at least one target agent. The shell may completely or partially encapsulate the core. In particular, the shell may completely cover the core. More in particular, the shell may be in contact with one portion of the core and at least one portion of the template.

The nanostructure may comprise at least one dimension having size ≦1000 nm. For example, ≦900 nm, in particular, ≦800 nm and even more in particular, less than 700 nm. More in particular, the nanostructure may comprise at least one dimension of size 500 to 600 nm. The sizes of the nanostructures increase the available surface area for a higher template loading. The small size of the nanostructures also enhances diffusion of small molecules into the core of the particles.

The binding site may be on the outer face of the shell for binding at least one target agent. In particular, the binding sites may be substantially on the face of the nanostructure. More in particular, most or all of the binding sites are on the outer face of the shell of the nanostructure. The presence of binding sites on the outer face of the shell may help to alleviate the issue of limited diffusion often associated with macromolecules. The nanostructures of the present invention may thus be used for binding of a large variety of target agents including macromolecules.

In particular, the binding site may fully envelope the target agent or the binding site may partially engulf the target agent. The target agent may be hydrophobic, hydrophilic or ampiphillic. In particular, the target agent may be hydrophobic such that the target agent may be completely in contact with the binding site or the target agent may be ampiphillic such that the target agent may partially interact with the binding site.

The nanostructure according to the invention may have a red blood cell morphology. More in particular, the red-blood cell morphology increases the surface area to volume ratio of the nanostructure enabling it to come in contact with a large amount of target agent, compared to spherical or substantially spherical nanostructures.

In particular, the target agent may be any component with a stable and/or static conformation to interact with the binding site. The target agent may be at least one hydrophilic drug, hydrophobic drug, vitamin, polysaccharide and/or steroid. The target agent may also be at least one protein, nucleic acid (like single or double strand DNA), virus, carbohydrate, macrocycle and/or a cell comprising at least one static conformation. The steroid may be cholesterol. The virus may be selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like. The target agent may be a full or a part of the virus. In particular, the part of the virus may be a protein coat (capsid), an envelope of fat and the like of the virus.

To further enhance the scope of potential applications of the core-shell nanostructure, the core may be magnetic. In particular, the core may comprise magnetic material. Any magnetic material known in the art suitable for the purpose of the present invention may be used. More in particular, Fe₃O₄ nanomagnetite may be encapsulated in the core-shell nanostructure, thus rendering the nanostructure superparamagnetic. For example, the magnetically susceptible imprinted nanostructures can be applied as an affinity adsorbent in protein purification to recognize and preferentially adsorb the protein of interest. The nanostructures may then be easily isolated by the use of an external magnetic field.

A strategy based on template immobilization and core-shell miniemulsion polymerization is developed as an improved method for protein imprinting. The creation of imprinted, or recognition, binding sites in polymers through specific 3-dimensional arrangements of monomers around the template molecules to be imprinted results in the recognition effect relying on interaction forces like ionic charges, hydrogen bonding and hydrophobic interactions. This makes the imprint highly specific in recognizing only the template molecule as its target, and this behaviour may be considered a chemical analog of biological antibodies.

The method of preparing at least one core-shell nanostructure comprising at least one binding site, for binding at least one target agent, comprises:
(a) providing at least one first hydrophobic polymer to form at least one core;
(b) providing at least one template to bind to the core, wherein the template comprises the conformation of at least one target agent;
(c) providing at least one second hydrophobic polymer to form a shell on the core and on a portion of the template; and
(d) removing the template, to form at least one core-shell nanostructure comprising at least one binding site, for binding the target agent.

A series of surface modifications may be carried out on the core particles to alter, change or modify its properties for template immobilization. For example, the surface modification may be carried out by adding at least one surfactant, lipid, polymer, inorganic material, or a mixture thereof. In particular, the surface modifications may include processes such as aminolysis, aldehyde functionalisation, plasma modification, ozonolysis, UV or radioactive degradation, hydrolysis, amide or ester bonding, click-chemistry reactions and the like.

With template immobilization, the issue of template solubility often encountered for protein imprinting through the traditional approach may be avoided. Such template immobilization strategy may allow the imprinting of proteins that may not be soluble in the polymerization mixture and can potentially be employed as a generally applicable methodology for protein imprinting. By using this approach, monodispersed surface-imprinted core-shell submicron particles with novel red-blood-cell (RBC)-like morphology may be prepared. The RBC-like morphology provides the imprinted nanostructures with large surface area to volume ratio for adsorption, thus allowing high template protein uptake and preserving the structure of the proteins. The creation of complementary binding sites on the particle outer face alleviates the difficulty of limited diffusion for protein macromolecules.

The binding site may be on the outer face of the shell for binding at least one target agent. In particular, the binding sites may be substantially on the face of the nanostructure. More in particular, most or all of the binding sites are on the outer face of the shell of the nanostructure.

The molecular imprinting may be based on a hydrophobic interaction system. In particular, recognition between the target agent and the binding site may be partly achieved based on shape and structure and partly based on binding between the target agent and the binding site. More in particular, recognition between the target agent and the binding site may be achieved based on shape and structure thus enabling molecular recognition of proteins in aqueous media for the first time. In particular, the recognition between the target agent and the binding site may be epitope independent.

The resulting high adsorption ratio, high separation factor of protein mixtures, and fast kinetics shows that this approach is suited for industrial applications.

The first and/or second hydrophobic polymer may be the same or different and may be selected from the group consisting of vinyl acrylate polymers, vinyl acetate polymers, acrylamides, nitrile polymers and/or a mixture thereof. In particular, the hydrophobic polymer may be selected from the group consisting of vinyl acrylate polymers, vinyl acetate polymers, acrylamides, acrylonitriles and/or a mixture thereof. More in particular, the hydrophobic polymer may be poly(methyl methacrylate), poly(ethylene glycol dimethacrylate), poly(methyl acrylate), poly(hydroxylethylmethacrylate), poly(vinyl acetate), poly(vinyl alcohol), poly(vinyl acrylamide), and other chain polymers that have hydrophobic functional groups such as alcohol, carboxyl, amide/amine and the like.

Mini-emulsion polymerization may be considered a flexible process enabling various polymers to be formed, and it may provide a convenient strategy for incorporating desired features (like magnetic susceptibility for example) into the imprinted nanostructures. For example, superparamagnetic nanostructures may be used for easy separation of the nanostructures from a mixture.

Complementary binding sites for the template protein molecules may be formed on the nanostructure outer face by synthesizing an external polymeric shell layer over the template-immobilized core particles with subsequent template removal by hydrolysis. In particular, alkaline hydrolysis or acidic hydrolysis may be used to remove the template.

In another aspect of the present invention, there is provided at least one core-shell nanostructure obtainable or obtained according to the method of any aspect of the present invention.

In one aspect of the present invention, there is provided a nanostructure for binding at least one virus, the nanostructure comprising at least one hydrophobic polymer, and at least one binding site on the outer face of the nanostructure for binding the virus. The virus may be selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like. The target agent may be a full or a part of the virus. In particular, the part of the virus may be a protein coat, an envelope of fat and the like of the virus. In particular, the conformation of the virus and/or part thereof may be complementary to the conformation of the binding site.

The virus imprinted nanostructures may be fabricated using polymerisation. In particular, a mini-emulsion polymerization system may be used to disperse monomers in a continuous phase and the dispersion may be stabilized with a surfactant or emulsifier. Cross-linkers may then be added slowly to the aqueous phase containing the surfactants. Template may then be added followed by initiators which may initiate the polymerization to form nanostructures. The template may then be removed to form nanostructures for binding at least one virus. The high polymerization rate and superior heat dispersal capability due to the low viscosity of the continuous phase throughout the whole reaction will be viable for large-scale industrial systems. The cross-linkers and initiators to be used are well known in the art.

The surfactant may be a non-ionic or an ionic surfactant. More in particular, the surfactant may be anionic, cationic and/or zwitterionic. For example the surfactant may be selected from the group consisting of but not limited to Perfluorooctanoate (PFOA or PFO), Perfluorooctanesulfonate (PFOS), Sodium dodecyl sulfate (SDS), ammonium lauryl sulfate, and other alkyl sulfate salts, Sodium laureth sulfate, also known as sodium lauryl ether sulfate (SLES), Alkyl benzene sulfonate, Soaps, or fatty acid salts, Cetyl trimethylammonium bromide (CTAB), hexadecyl trimethyl ammonium bromide, and other alkyltrimethylammonium salts, Cetylpyridinium chloride (CPC), Polyethoxylated tallow amine (POEA), Benzalkonium chloride (BAC), Benzethonium chloride (BZT), Dodecyl betaine, Cocamidopropyl betaine, Coco ampho glycinate, Alkyl poly(ethylene oxide), Alkylphenol poly(ethylene oxide), Copolymers of poly(ethylene oxide) and poly(propylene oxide) (commercially called Poloxamers or Poloxamines), Alkyl polyglucosides, including Octyl glucoside, Decyl maltoside, Fatty alcohols, Cetyl alcohol CA), Poly(vinyl alcohol)(PVA), Oleyl alcohol, Cocamide MEA, cocamide DEA, Polysorbates such as Tween 20, Tween 80 and Dodecyl dimethylamine oxide. When the target agent is a large molecule, for example, a protein, virus, cell and the like thereof, the surfactant may be SDS, CA and/or PVA. In particular, at least one surfactant may be used in the process for the preparation of the nanostructures. The concentration of the surfactants may be in the range of 0.005 to 0.3M. In particular, 0.01 to 0.2M, 0.01 to 0.1M, or 0.015 to 0.03M. In one aspect, at least two surfactants may be used in the process for the preparation of the nanostructures according to the invention in a ratio of 1:1 to 1:10 w:w PVA:SDS or PVA:CA.

In particular, the ratio of the surfactants may be 1:3, 1:5 or 1:8. More in particular, the ratio may be 1:3. According to a particular aspect, solvent is not used in the preparation of the nanostructures of the invention, particularly when the template/target is a large molecule. Water may be used instead.

This polymerization system may give highly regular and mono-dispersed polymeric particles of sizes between 50-500 nm. In particular, at least one dimension of the nanostructure may be 500 nm in size. More in particular, at least one dimension of the nanostructure may be 450 nm, 400, 350 nm, 300 nm, 250 nm, 200 nm, 150 nm, 100 nm or, 50 nm in size.

Monomers used may be selected from the group consisting of but not limited to hydrocarbons such as the alkene and arene homologous series. In particular, monomers may be phenylethene, ethane, acrylic monomers and the like. For example, methyl methacrylate, MMA may be used as the monomer.

According to another aspect, the nanostructure of the present invention may be for use as antibody substitute. In particular, the antibody substitute may be synthetic, non-toxic and biocompatible. The polymers of the antibody substitute may sequester away the viruses to prevent them from infecting cells, eliminating the need for any immune response. This may render the viruses ineffective as they may be tightly bound to the polymers which can later be excreted via the kidneys. Modifications to the nanostructures in any aspect of the present invention with encapsulated nanosilver or titanium dixoide may subsequently be made to deactivate captured viruses. Small mutations of viral DNA may also not be an issue, when using the nanostructures according to any aspect of the present invention as the target for recognition is broader based than just one protein of the virus. The synthesis of the nanostructure according to any aspect of the present invention may be considered simple, scale-up and large quantity production may be easy, and thus the approach may be comparatively very low cost.

According to a further aspect, the present invention provides a method of imaging of at least one subject, the method comprising:
(a) administering the nanostructure according to any aspect of the present invention to at least one subject;
(b) allowing the nanostructure to contact the target agent to form at least one nanostructure-target agent complex; and
(c) detecting the presence of the nanostructure-target agent complex in the subject.

According to another aspect, the present invention provides a method of detecting and/or imaging at least one target agent in at least one biological sample, the method comprising:
(a) collecting at least one biological sample from a subject;
(b) contacting (administering) the nanostructure according to any aspect of the present invention to the biological sample;
(c) allowing the nanostructure to contact the target agent to form at least one nanostructure-target agent complex; and
(d) detecting the presence of the nanostructure-target agent complex in the biological sample of the subject.

The biological sample may be any biological fluid obtainable from a subject. For example, but not limited to, blood, serum, spinal fluid, saliva and/or urine. The core-shell nanostructure may further comprise at least one label which is detectable when the core-shell nanostructure may be bound to the target agent. A label is a chemical, moiety or molecule that allows detection of the label together with any molecule, surface or material to which the label is applied, attached, coupled, hybridized and/or bound to. Examples of labels include but are not limited to dyes, radiolabels, fluorescent labels, magnetic labels and enzymatic labels. In particular, the label may be a reporter molecule that may be activated when the target agent binds to the nanostructure.

According to one aspect the present invention provides a method of diagnosis of at least one disorder, the method comprising:
(a) collecting at least one biological sample from a subject;
(b) contacting (administering) the nanostructure according to any aspect of the present invention to the biological sample;
(c) allowing nanostructure to contact the target agent to form at least one nanostructure-target agent complex; and
(d) detecting the presence of the nanostructure-target agent complex in the biological sample;
   wherein detection of the nanostructure-target agent complex indicates the presence of the disorder in the subject.

The biological sample may be any biological fluid obtainable from a subject. For example, but not limited to, blood, serum, spinal fluid, saliva and/or urine. In particular, the disorder may be at least one viral infection. More in particular, the viral infection may be a result of the virus selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like.

The target agent may be at least one be at least one infected cell. In particular, the cell may be infected with any infectious disease. For example, the infectious disease may be but not limited HIV/AIDS, TB, malaria, HBV, HCV, pertussis, poliomyelitis, diphtheria, measles, tetanus and the like.

According to another aspect of the present invention there is provided a method for selective binding, separation, and/or purification of at least one target agent from a mixture of agents, wherein the mixture of agents comprises the target agent and at least one non-target agent and wherein the method comprises:
(a) contacting the nanostructure according to any aspect of the present invention to a mixture of agents;
(b) allowing the binding of the nanostructure to the target agent in the mixture of agents to form at least one nanostructure-target agent complex;
(c) separating the nanostructure-target agent complex from the mixture of agents; and
(d) separating the target agent from the nanostructure-target agent complex to obtain the target agent.

The nanostructures have a very high separation efficiency in a competitive environment in which only the pure templated protein may be adsorbed while the other proteins are left in solution.

According to one aspect of the present invention there is provided, a method of treatment of at least one disorder in a subject, the method comprising, administering the nanostructure according to any aspect of the present invention to the subject with the disorder.

The disorder may be at least one viral infection. In particular, the viral infection may be a result of the virus selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like.

According to one aspect, the present invention provides a use of the nanostructure according to any aspect of the present invention for the preparation of a medicament for the treatment of at least one disorder. The disorder may be at least one viral infection. In particular, the viral infection may be a result of the virus selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like.

According to another aspect, the present invention provides a nanostructure according to any aspect of the present invention for use in the treatment of a disorder. The disorder may be at least one viral infection. In particular, the viral infection may be a result of the virus selected from the group consisting of Retroviruses, Togaviruses, Filoviruses, Herpesviruses, Arenaviruses, Pox viruses, Coronaviruses, Rhobdoviruses, Paramyxoviruses, Orthomyxoviruses and the like.

According to another aspect, the present invention provides a pharmaceutical composition comprising the nanostructure according to any aspect of the present invention. The pharmaceutical composition may further comprise at least one pharmaceutically acceptable excipient, diluent, carrier and/or adjuvant.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

### Synthesis of the BSA Surface Imprinted Particles

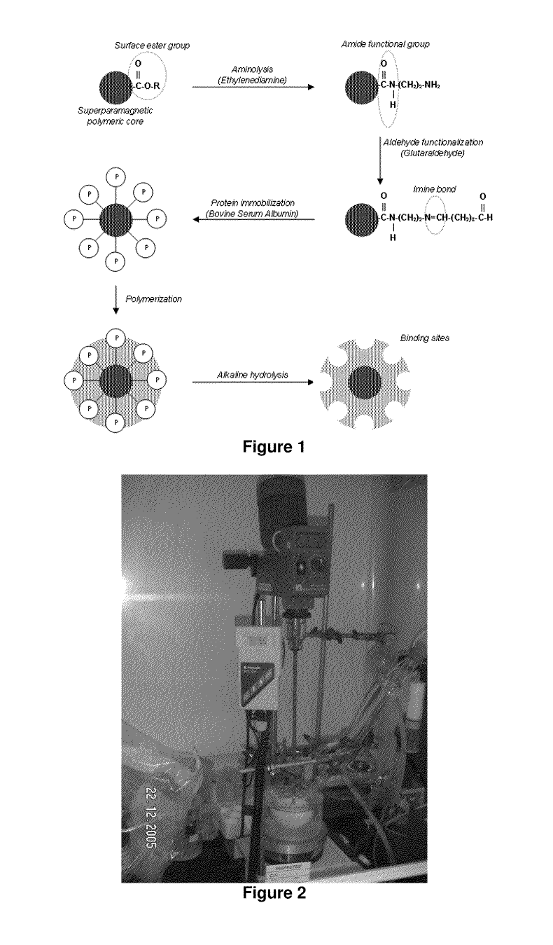

BSA surface-imprinted particles had been successfully synthesized with a two-stage core-shell miniemulsion polymerization. The imprinting strategy was based on the surface immobilization of template BSA molecules with a series of surface modification of the support beads prior to polymerization (FIG. 1). The miniemulsion polymerization was carried out using the set-up as shown in FIG. 2.

### Materials

Bovine serum albumin was used as the template protein, while lysozyme (Lys) from chicken egg white was used as the non-template (control) protein. Both proteins, sodium bisulfite (minimum 99%), and glutaraldehyde (50%) were purchased from Sigma. MMA (99%), EGDMA (98%), oleic acid (90%), sodium dodecyl sulfate (SDS; minimum 98.5% GC), sodium bicarbonate (99.7-100.3%), sodium bisulfite (minimum 99%), ammonium persulfate (APS, 98%), hydrochloric acid, cetyl alcohol (CA, 95%), ethylene diamine (EDA, 99%), and trifluoroacetic acid (TFA, 99%) were purchased from Aldrich. Ammonia solution (25%), ethanol, N,N-dimethylformamide (DMF), iron(II) chloride tetrahydrate (FeCl₂.4H₂O), and iron(III) chloride hexahydrate (FeCl₃.6H₂O) were obtained from Merck. Sodium hydroxide pellets were from J. T. Baker, acetic acid from Fisher Chemicals (UK), and HPLC-grade acetonitrile from Tedia. All chemicals were used directly without further purification.

In the present examples, CA and SDS are used as surfactants. Alternatively, PVA may be used instead of SDS or of CA.

### Preparation of Fe₃O₄ Magnetite

Fe₃O₄ magnetite was prepared by a co-precipitation method (Liu et al., 2005). A 25-mL mixture containing 0.8 M FeCl₃.6H₂O, 0.4 M FeCl₂.4H₂O, and 3 vol % concentrated hydrochloric acid was prepared in deionized (DI) water. The resulting clear yellowish green solution was then added into 250 mL of a 5.23 vol % ammonia solution. Upon addition, the solution turned black and was then stirred magnetically at 1000 rpm for 1 h. The Fe₃O₄ magnetite was then washed three times with Deionised (DI) water before being suspended in the water.

### Preparation of Superparamagnetic Support Particles (Shiomi et al. (2005) and Bonini et al. (2007).

One gram of the Fe₃O₄ magnetite prepared above was mixed with 1.0 mL of oleic acid to obtain a black viscous gel. MMA (1.28 mL) and EGDMA (9.05 mL) in the molar ratio of 1:4 were then added to the oleic acid-coated magnetite and mixed thoroughly. The mixture was then ultrasonicated at 65% power level for 80 s (Sonics Vibracell VCX 130, Sonics & Materials Inc., Newtown, Conn.). After homogeneity was achieved, the resulting mixture was added dropwise into a 50-mL solution of 0.01 M SDS and 0.03 M CA, which was magnetically stirred at 300 rpm. The mixture was further ultrasonicated at 65% power level for 90 s to create a miniemulsion. The miniemulsion was then added dropwise into 600 mL of a 0.05 w/v % SDS solution. This reaction mixture was transferred to a 1-L, three-neck, round-bottom flask and purged with nitrogen gas for 15 min to displace oxygen while maintaining the temperature at 80° C. Subsequently, APS (0.5 g) was added to the reaction mixture to initiate the polymerization reaction. The reaction was allowed to proceed for 24 h. Upon completion, the polymeric support beads were washed three times with DI water, three times with 50 vol % ethanol, and finally, three times with DI water.

The superparamagnetic Fe₃O₄ magnetite nanoparticles were first prepared using the coprecipitation method. Previous measurements of the particles by FESEM showed that their sizes were ˜18 nm. The magnetite gel was made hydrophobic with a coating of oleic acid, which helped to enhance the penetration of the magnetite into the hydrophobic interior of micelles during the first-stage core-shell miniemulsion polymerization. This strategy was successful in the fabrication of the magnetically susceptible support polymeric beads. MMA has been chosen as the monomer for this preparation. It is a common monomer used for the oil-in-water (o/w) miniemulsion polymerization and also is commonly used in molecular imprinting through hydrophobic interactions. In addition, it is able to provide ester and methoxy groups for subsequent surface functionalization. Being a weak electron donor, the esters groups are susceptible to nucleophilic attack during the aminolysis substitution reaction. The addition of EGDMA as a cross-linker maintained the stability of the imprinting sites while making the product polymeric beads easier to be handled and processed.

Surface Modifications of the Support Particles-Aminolysis

One gram of the polymeric support particles prepared above was washed twice with DMF and redispersed in 20 mL of DMF. Subsequently, 20 mL of EDA was added to the mixture and magnetically stirred at 400 rpm for a 12-h reaction under reflux at 110° C. The amine-functionalized core particles were then washed once with DI water, twice with 50 vol % ethanol, and finally, twice with DI water.

Surface Modifications of the Support Particles-Aldehyde Functionalization

A buffer solution of pH 5 was prepared using acetic acid and NaOH. One gram of the amine-functionalized polymeric support particles prepared above was soaked in 10 mL of buffer solution and degassed for 10 min at room temperature. The buffer was then removed, and the particles were redispersed in 10 mL of fresh buffer with 5 vol % glutaraldehyde. This mixture was allowed to react with magnetic stirring at 400 rpm for 12 h at room temperature. The aldehyde-functionalized particles were then washed three times with DI water post-treatment.

Immobilization of Template BSA

The aldehyde-functionalized polymeric particles prepared above were washed once with 0.01 M phosphate buffer saline (PBS). A 10-mL aliquot of BSA solution (2.5 mg/mL) was then added to 1.0 g of the particles. The mixture was magnetically stirred at 300 rpm for 3 h at 4° C. for the coupling to occur. The BSA-immobilized core particles were then washed three times with DI water upon completion of the reaction. Upon completing the post synthesis processing of the support particles, they were then subjected to a series of surface functionalization reactions as illustrated in FIG. 1. XPS was employed as the primary tool to monitor the reactions. It is routinely applied for characterizing surface modifications and has been found to be able to provide insights on the surface information of a material. Elemental wide scans were conducted and the surface atomic compositions are reported in Table 1 as provided below. Fourier transform infrared (FT-IR) spectroscopy was also considered as an alternative probe for the purpose but was found to be not suitable because FT-IR examined the bulk composition of the particles rather than just the surface. The success of each surface modification reaction could be associated with changes in the surface composition of nitrogen atoms. The increase in the nitrogen atomic composition from 0.00 to 0.87% after the first aminolysis functionalization suggested that amine groups were successfully introduced onto the surfaces of the polymeric support particles. An activated carboxylic acid derivative is usually required for the aminolysis reaction, which involves nucleophilic acyl substitution. Nevertheless, MMA and EGDMA are capable of providing activated yet thermally more stable surface ester groups for the nucleophilic acyl substitution reaction.

The amine-modified surface was subjected to further reactions to introduce aldehyde groups. Glutaraldehyde was chosen as the bridging agent as it possesses two terminal aldehyde groups. As one of the aldehyde groups was reacted with the amine groups on the polymeric support particle surface, the other was left free. Subsequently, under the suitable conditions, the free surface aldehyde was reacted with free amine groups in the template BSA molecules (from lysine, for example), thus successfully immobilizing BSA molecules onto the support particle surfaces. Both reactions involved nucleophilic addition that allowed the formation of imine bonds between an aldehyde and an amine groups. The reactions are reversible and acid catalyzed at an optimum pH of 5.

After the aldehyde functionalization, the decrease in the nitrogen composition from 0.87 to 0.60% and an accompanying increase of carbon composition from 75.52 to 79.63% might be indicative of the relative increase in carbon and oxygen content from glutaraldehyde. Although the deconvolution procedure is not exact, it provided some insights on the type of functional groups that could be found on the particle surface. Thus, the success of BSA immobilization can be seen by the significant increase in the nitrogen composition from 0.60 to 2.73% through XPS analysis. This increase was attributed to the abundant peptide bonds from the protein molecules.

TABLE 1
Surface Atomic Compositions of the Support
Particles from the XPS Widescan Spectra

| | elemental atomic composition | | |
|---|---|---|---|
| stage | C | O | N |
| core surface | 80.04 | 19.88 | 0.00 |
| NH₂ functionalization | 75.52 | 23.51 | 0.87 |
| CHO functionalization | 79.63 | 19.66 | 0.60 |
| protein immobilization | 76.05 | 21.00 | 2.73 |
| after alkaline hydrolysis | 69.11 | 29.33 | 0.95 |

In this series of surface functionalization reactions, after modifying the particle surface with amine moieties, the template BSA molecules was coupled to the particle surfaces through bridging glutaraldehyde molecules instead of direct coupling via an amide bond. This is to prevent any couplings between the protein molecules. To ensure the success of the functionalization reactions, the products of each modification step were analyzed by XPS with the C1s and O1s spectrums deconvoluted for further analysis. The observed carbon ratio obtained for the unmodified support beads that contain surface ester groups is in good agreement with the theoretical ratio. The experimental carbon ratios for the amine- and aldehyde-functionalized support particles were lower than the expected ratio for 100% conversion, and these experimental ratios were unable to provide conclusive evidence on the conversion yield.

Imprinted Shell Layer Synthesis

An external imprinted polymeric shell was created over the BSA-immobilized support beads during the second-stage miniemulsion polymerization with MMA and EGDMA as the functional and cross-linking monomers, respectively.

MMA (1.28 mL) and EGDMA (9.05 mL) were mixed with 1.0 g of the surface-modified superparamagnetic core particles. The mixture was then ultrasonicated at 45% power level for 90 s to ensure that it was thoroughly mixed. It was then added dropwise into a 50-mL solution of 0.01 M SDS and 0.03 M CA, which was stirred at 300 rpm. The mixture was ultrasonicated again at 45% power level for 110 s to generate the miniemulsion. The resulting miniemulsion was then added dropwise into 600 mL of a 0.05 w/v % SDS solution and was stirred at 300 rpm. This mixture was subsequently transferred to a 1-L, three-neck, round-bottom reactor and purged with nitrogen gas for 15 min at 40° C. to displace oxygen. Sodium bisulfite (0.25 g) followed by APS (0.25 g) was then added into the mixture to initiate the polymerization reaction, which was allowed to proceed for 24 h. Upon completion, the polymeric core-shell particles were washed three times with DI water, three times with 50 vol % ethanol, and finally, three times with DI water.

Template Removal Hydrolysis

After the formation of the shell layer, the immobilized template BSA molecules were removed by hydrolysis. A 10-mL aliquot of a 1.0 M NaOH solution was added to 1.0 g of the core-shell particles. The hydrolysis mixture was stirred at 300 rpm and allowed to react for 5 h under reflux at 35° C. These surface-imprinted particles (iMIP) were washed three times with DI water and resuspended in DI water for characterization and adsorption studies and for storage.

The BSA-surface imine linkage was hydrolyzed to remove the template BSA, leaving behind complementary binding sites on the particle outer shell. The ease of hydrolyzing the imine bond was the primary reason for its use in this work with oxalic acid and sodium hydroxide being the common catalysts used for this reaction. An initial attempt was made to remove the template by acid hydrolysis; however, this resulted in the dissolution of the iron oxide in the core particles; hence alkaline hydrolysis was employed instead. The successful removal of the BSA molecules was verified by the significant reduction of nitrogen composition (Table 1) and the disappearance of the N1s peak from the XPS wide scan spectra (results not shown). A corresponding change was not observed for the nonimprinted particles (iNIP). Furthermore, there were no significant differences between the surface elemental composition of the iMIP and iNIP (results not shown). This further verified the success of the template removal. Other than the imprinted particles based on immobilized template molecules (iMIP), three other types of particles, namely, nonimprinted particles with similar surface functionalization (iNIP), imprinted particles with non-immobilized (or free) template molecules (fMIP), and nonimprinted particles without the surface modification (fNIP), had also been prepared. These particles were used as control samples for subsequent characterization studies.

Preparation of Nonimprinted Particles from Surface-Modified Support Beads (iNIP)

The corresponding nonimprinted particles to the above iMIP were prepared using steps similar to those above, except without the surface immobilization of BSA templates before polymerization of the external shell layer. These particles were used as control samples for comparison in the characterization studies.

Preparation of Molecularly Imprinted Particles from Unmodified Core Beads Using Free Template (fMIP)

Magnetically susceptible molecularly imprinted polymers using free (nonimmobilized) BSA template were also prepared. The magnetically susceptible polymeric support beads were prepared as above. However, no further surface modification reactions were carried out except for the following shell polymerization. Twenty-five milligram of BSA was first dissolved in 10 mL of DI water. MMA (1.278 mL), EGDMA (9.054 mL), and 10 mL of the prepared BSA solution were then added to 1.0 g of the superparamagnetic core particles. Subsequently, the resulting mixture was ultrasonicated at 45% power level for 90 s. A brown viscous mixture was obtained, which was then added dropwise to a 50-mL solution of 0.01 M SDS and 0.03 M CA. The mixture was then ultrasonicated at 45% power level for 110 s to produce the miniemulsion. The miniemulsion was added dropwise to a 600 mL of 0.05 w/v % SDS solution before being transferred to a 1-L, three-neck, round-bottom flask. This mixture was purged with nitrogen to displace oxygen and was heated to 40° C. Sodium bisulfite (0.25 g) followed by APS (0.25 g) was then added to initiate the reaction. With the temperature maintained at 40° C., the mixture was mechanically stirred at 300 rpm and the polymerization reaction was allowed to proceed for 24 h. Upon completion of the reaction, the fMIP were washed twice with DI water, three times with a solution of 10 w/v % SDS to 10 v/v % acetic acid, three times with 50 vol % ethanol, and finally, three times with DI water for template removal.

Preparation of Nonimprinted Particles from Unmodified Core Beads (fNIP)

A corresponding control sample to the above fMIP was prepared using a similar method except without the addition of the template BSA protein in the miniemulsion.

Analysis and Measurement

XPS (AXIS His-165 Ultra, Kratos Analytical, Shimadzu) was employed to determine the surface elementary composition of the support particles at each stage of surface modification.

Size Measurements

The sizes of the polymeric particles were determined using LLS (BIC Particle Sizing Software 90 Plus, Brookhaven Instruments Corp.).

The sizes of the support beads, iMIP, iNIP, fMIP, and fNIP, were determined using LLS and the results are as tabulated in Table 2. From the measurement, it was found that the particles were monodispersed in size. The support beads sized ˜350 nm while the mean effective diameters of the other four particles ranged from 500 to 600 nm. The larger sizes of the particles suggested a successful shell formation over the core beads.

TABLE 2
Morphological Features of the Polymeric Particles Prepared

| | mean effective | poly- | swelling |
|---|---|---|---|
| polymer | diameter (nm)^{a} | dispersity | ratio |
| support beads | 352.8 | 0.141 | |
| iMIP | 535.2 | 0.005 | 3.58 ± 0.78 |
| iNIP | 580.9 | 0.006 | 2.73 ± 0.53 |
| fMIP | 603.4 | 0.005 | 2.47 ± 0.32 |
| fNIP | 489.6 | 0.060 | 2.41 ± 0.56 |

^{a}Results obtained from LLS measurements.

Morphological Observations

Morphological observation of the polymeric particles was performed with a FESEM (JSM-6700F, JEOL) and a TEM (JEM-2010, JEOL). TGA (TGA 2050, TA Instrument) was employed to determine the efficiency of the magnetite encapsulation within the polymeric particles.

FESEM and TEM were employed to observe the morphological features of the particles. From the FESEM images (FIG. 3a), the polymeric support particles appear to be spherical in shape. Through TEM observation, due to a difference in the densities of the copolymer and the iron oxide, the magnetite is seen as the darker spots inside the support beads (FIG. 3d). This illustrates the successful encapsulation of the magnetite into the core particles. Being different from the support core particles, the iMIP and iNIP were monodispersed with a unique “red blood cell” (RBC)-like morphology (FIG. 3b and c) and there were no significant morphological differences between all of the particles (fMIP and fNIP also had similar morphological features, results not shown).

A reduced amount of monomers had been used in the second-stage polymerization reaction for the fabrication of the RBC-like core-shell particles. With this structure, the external shell created (the concave morphology) would be close to the core particle surface and, hence, enabling the formation of imprinted binding sites near the product core-shell particle surface. After the second-stage miniemulsion polymerization, the immobilized template BSA molecules could have been covered by the polymeric shell layer. Even so, with the unique concave morphology, the binding sites created for BSA would still be very close to the surface and thus the template removal through base hydrolysis would not face any hindrances.

In addition to that, as seen from Table 2, the particles sizes did not change significantly after the second-stage polymerization and this further substantiates the presence of the BSA binding site near the surface. It is well-known that the concave shapes of red blood cells provide maximum surface area per unit volume, thus facilitating gas transfer into and out of the cells. Similarly with the RBC-like morphology, the core-shell imprinted particles possessed high specific surface area for effective template uptake during adsorption processes. In fact, the thickness of a polymeric shell layer can also be controlled through the application of a controlled polymerization technique such as surface-initiated atom-transfer radical polymerization. However, the strategy used here to produce RBC-like particles proved to be relatively simpler and is a more convenient alternative as fewer complications were involved.

### Batch Rebinding Tests

In characterizing the adsorption behaviours of the core-shell particles, they were subjected to batch rebinding, competitive rebinding, and adsorption kinetics studies.

The initial BSA concentrations of the adsorption samples varied from 1.2 to 2.0 mg/mL. The samples were affixed onto a Rotamix (RKVS, ATR Inc.) and agitated by end-to-end rotary mixing for 24 h at room temperature. The amount of protein adsorbed by the polymeric particles at the end of each run was determined by the following formula:

$$Q = \frac{( {C}_{i} - {C}_{f} ) V}{m}$$

where Q (mg of protein/g of polymer) is the mass of protein adsorbed per gram of polymer, Cᵢ (mg/mL) is the initial protein concentration, C_{f} (mg/mL) is the final protein concentration, V (mL) is the total volume of the adsorption mixture, and m is the mass of polymer in each rebinding mixture. The final concentration, C_{f}, was determined by using an Agilent 1100 series HPLC unit with an Agilent Zorbax 300SB-C18, 4.6×150 mm, 5-μm reversed-phase column. At the end of 24 h, the samples were centrifuged (Universal 32R, Hettich Zentrifugen) at 9000 rpm for 40 min in order to extract the supernatants, which were prefiltered using sterile 0.2-μm filter units and subsequently analyzed by HPLC. Two mobile phases, (A) ultrapure water with 0.1 vol % TFA and (B) 80 vol % acetonitrile and 20 vol % water with 0.09 vol % TFA, were used for the linear gradient elution. The solvent flow rate was set at 1 mL/min with solvent B increasing from 25 to 70 vol % in 40 min. The analyte injection volume was 50 μL, and the column temperature was set at 60° C. The samples were analyzed by an UV detector at a wavelength of 220 nm. For a comparative assay, the iNIP, fMIP, and fNIP were also subjected to the batch rebinding test. Similar tests had also been carried out with the non-template Lys because Lys is much smaller size than BSA. All tests were conducted in triplicates.

The imprinted sites created for BSA will thus not be able to keep the competitor Lys out based on size exclusion. Hence, any preferential uptake of BSA over Lys will be a strong indication of the molecular imprinting effect. Nevertheless, there may be concerns over the suitability of Lys as a competing protein due to its significantly different isoelectric point from that of BSA. In many cases, monomers such as MAA and acrylamide have been applied for protein imprinting due to their favourable hydrogen bond formation and electrostatic interactions with template protein molecules. In order to achieve the desired molecular affinity for the template BSA in an aqueous environment, the synergistic effect of hydrophobic interactions and shape complementarity were used instead. Thus, the rather hydrophobic MMA was used for particle fabrication and thus will result in reducing the effects of the acidity/basicity of proteins on the recognition and rebinding processes in this system. It is hypothesized, that protein-imprinting is thus due solely to these hydrophobic interactions.

As shown in FIG. 4a, the iMIP (as shown in black blocks) exhibited significantly higher BSA loadings than the counterpart control iNIP (as shown in white blocks) for all different initial concentrations, with the highest loading of 854 nmol/g at the initial concentration of 1.8 mg/mL. This is a proof of the successful creation of imprinted cavities on the iMIP. The BSA adsorption capacity ranges from 40 to 100 nmol/g. It can be seen that the iMIP obtained here displayed a significantly higher BSA loadings. It is hypothesized that this is due to the RBC-like morphology of the imprinted particles with its high surface area to volume ratio.

Furthermore, the BSA loadings of iMIP were also generally higher than that for the fNIP (as shown in broken crosshatch blocks) and fMIP (as shown in crosshatch blocks). Although the shell layers of fMIP had been created in the presence of non-immobilized BSA templates, they did not consistently adsorb more BSA than the control fNIP in the batch rebinding tests. In fact, the BSA loadings for fNIP and fMIP were not significantly different, illustrating the poor imprinting efficiency with the use of the free template strategy.

When the test was conducted with Lys (FIG. 4b), the Lys uptake of all the particles was random with no conclusive trend to be drawn. This was expected since Lys was the non-template protein and its adsorption was attributed to be from non-specific interactions. Similarly, as Lys is smaller than BSA, more Lys molecules could thus adsorb non-specifically onto the material causing the Lys loadings of the particles as generally higher than BSA as observed. Despite this, the significantly higher BSA uptake by iMIP compared to other particles (iNIP, fNIP, fMIP), which was not observed for the case of the non-template Lys, is a convincing indication of the recognition property imparted through molecular imprinting.

Based on the amount of BSA adsorbed (O) at the initial concentration of 1.8 mg/mL, the imprinting efficiency had been calculated and the results are presented in Table 3. It is shown that the iMIP achieved an imprinting efficiency of 6.51 while the fMIP imprinting efficiency is only 0.94. This demonstrated the recognition property of the iMIP and the importance of template immobilization for the imprinting process.

TABLE 3
Results Obtained from the Batch Rebinding Tests

| polymer | Q at 1.8 mg/mL (nmol/g) | imprinting efficiency^{a} |
|---|---|---|
| iNIP | 131.98 | — |
| iMIP | 859.21 | 6.51 |
| fNIP | 358.26 | — |
| fMIP | 335.29 | 0.94 |

^{a}Imprinting efficiency = Q (for imprinted particles)/Q (for non-imprinted particles)

Competitive Batch Rebinding Tests

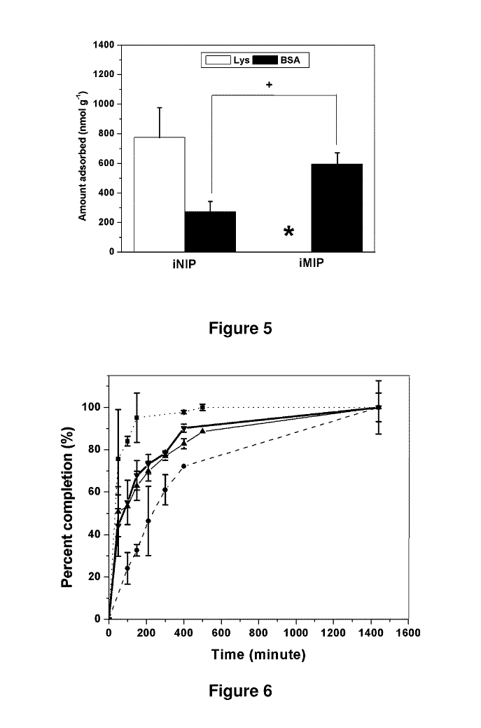

To further illustrate the recognition property of the iMIP, the core-shell particles were subjected to binary protein competitive assay where, similarly, Lys had been employed as the competitor protein. The polymeric particles were subjected to a binary protein mixture of BSA and Lys with individual initial concentrations of 1.8 mg/mL. The adsorption mixture was rotary mixed for 24 h and analyzed similarly as in the batch adsorption experiments above. All of the competitive batch rebinding tests were conducted in triplicate. The results are shown in FIG. 5.

The iNIP adsorb more Lys than BSA while iMIP had not only exhibited a higher uptake of BSA than Lys in the competitive system, the adsorption of the non-template Lys had been effectively suppressed. In a competitive environment of protein adsorption, the adsorbent surface is usually first occupied by smaller proteins, which have higher diffusion coefficients. Nevertheless, at later stages, the already adsorbed proteins will be displaced by proteins (in this case, BSA) that have greater affinity toward the adsorbent surface. This is known as the Vroman effect and is probably responsible for the effective suppression of Lys adsorption observed. The results indicated the molecular affinity of iMIP for the template BSA molecules. In addition, the iMIP displayed a significantly higher BSA loading (595 nmol/g) than the iNIP (273 nmol/g) in the binary protein system. In this case, the BSA uptake of the iMIP was significantly reduced as compared to that observed in the single-protein adsorption systems (batch rebinding tests). This was nevertheless expected and was attributed to the adsorption competition from the second protein. When fNIP and fMIP were subjected to a similar competitive assay (results not shown), the fMIP did not exhibit a preferential uptake of the template BSA over its corresponding nonimprinted iNIP. Instead, the two types of particles displayed similar BSA and Lys loadings. This further illustrated the poor imprinting efficiency for the fMIP where non-immobilized BSA had been employed in the molecular imprinting process.

Adsorption Kinetics Study

The adsorption kinetics of the particles prepared was studied with an initial BSA concentration of 1.8 mg/mL. The adsorption runs were performed similarly to the single-protein batch rebinding tests. To determine the adsorption profiles of the samples, analytes were drawn at regular intervals for HPLC analysis to determine the BSA concentrations. The tests were conducted in triplicate.

The adsorption kinetics of proteins is one of the important considerations for the practical application of molecularly imprinted particles. The rebinding kinetics of BSA to the particles was therefore studied in this investigation. The results obtained in terms of percentage completion are shown in FIG. 6. In general, the observed rebinding curves for all samples are typical as in most adsorption processes, having a relatively high initial adsorption rate that decreases slowly over time to finally achieve equilibrium. It was observed that there was no significant variation between the rebinding kinetics for the fMIP (as shown in black inverted triangles) and fNIP (as shown in black upright triangles). This showed that there were no differences in the particles as adsorbents for BSA, thus indicating that the use of free template molecules for surface imprinting was inefficient. For the iNIP (as shown in black squares), the adsorption kinetics was favorable, reaching equilibrium (>95% completion) in ˜150 min. On the other hand, despite a display of significant molecular selectivity in the batch and competitive rebinding tests, the iMIP (as shown in black circles) had surprisingly slower kinetics as compared to iNIP. For the iNIP, the template adsorption could be nonspecific, while for the iMIP, more time would probably be required for the template molecules to orient themselves to specifically fit into the imprinted cavities. This hypothesis provided a possible explanation for the slower rebinding kinetics observed in the iMIP. Furthermore, the BSA loadings of the nonimprinted particles were less than their imprinted counterparts, thus probably enabling the equilibrium to be achieved within a shorter period of time.

Statistical Analysis

Standard deviation calculations and Student's t-test were carried out using Microsoft Excel (Seattle, Wash.) for statistical comparisons between pairs of samples. The groups were considered statistically different when p<0.05.

### Example 2

### Synthesis of the Virus Imprinted Nanostructures

A virus particle is a gene transporter that contains the most basic level of nucleic acids surrounded by a protective coating known as capsid. A capsid is composed of proteins encoded by the viral genome and its shape will therefore serve as the basis for its morphological imprinting. The template virus to be used here is a simple bacteriophage (M13 containing luciferase gene infecting E. coli). Viral capsid and surface proteins will be characterized using SEM/TEM, MALDI-TOF-MS, LC-MS/MS and x-ray crystallography.

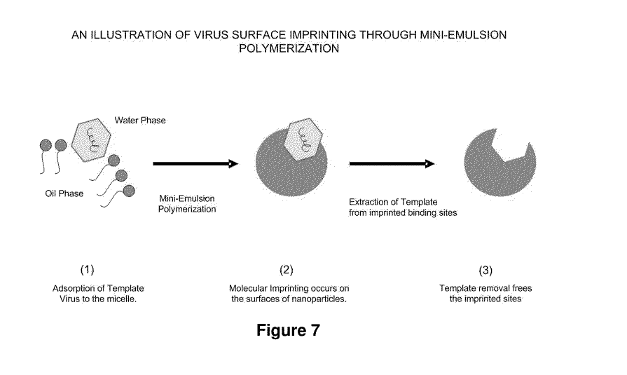

The virus imprinted nanostructures will be fabricated using a mini-emulsion polymerization system which involves the dispersion of monomers in a continuous phase and the stabilization of this dispersion by a surfactant or emulsifier. This polymerization system is known to give highly regular and mono-dispersed polymeric particles of sizes between 50-500 nm. With its high polymerization rate and superior heat dispersal capability due to the low viscosity of the continuous phase throughout the whole reaction, it will also be viable for large-scale industrial systems. Briefly, monomers (methyl methacrylate, MMA) and cross-linkers (ethylene glycol dimethacrylate, EGDMA) will be added slowly to an aqueous phase containing the surfactants. After homogenization to create the mini-emulsion, the template virus (bacteriophage) will be added. Initiators will then be added and polymerization will proceed to form nano-particles. After fabrication, the MIPs will be washed to remove the template virus for further reuse. Non-imprinted nanoparticles (NIPs) without the template virus will also be prepared using the same protocol for control purposes. Subsequently, the nanoparticles will be characterized by field emission scanning electron microscopy (FE-SEM) and light scattering to determine the morphology and size. The hypothesized process of the virus surface imprinting is shown in FIG. 7.

Virus Rebinding Studies

In order to determine the effectiveness of the MIPs in adsorbing and removing the viruses, rebinding studies will be performed. Both the imprinted and non-imprinted nanoparticles will be subjected to this rebinding test. Solutions of pure viruses at different concentrations will be mixed with the MIPs for 24 hours. After removing the MIPs, the concentration of viruses remaining in solution will provide the binding efficiency. The MIPs bound viruses will also be characterized for bioactivity by determining its ability to infect E. coli with luciferase. The concentration and kinetics of viral adsorption will be measured with a high performance liquid chromatograph (HPLC). As controls, non-template viruses (e.g. plant tobacco mosaic virus) will be used in competitive rebinding assays.

Anti-Viral Studies

Viruses depend on the host cells that they infect to reproduce. Bacteriophage virus used as the template, can infect specific bacteria by binding to surface receptor molecules and then entering the cell. Within a short amount of time, viral DNA can be translated into proteins that eventually become either new virions within the cell, helper proteins which help in assembly of new virions, or proteins involved in cell lysis resulting in the release of more phages.

The prevention of viral infection caused by the M13 phage will be analyzed with bacteria. E. coli bacteria will be cultured and expanded in a standard culture media for 72 hours. The synthesized MIPs will then be added to the media and thoroughly mixed. Bacteriophage with a luciferase reporter gene will subsequently be added to the mixture in the attempt to “infect” the bacteria. This gene encodes light producing enzyme, luciferase that serves as a marker for gene activation. Any virus not captured by the MIPs will have the ability to transfect the bacteria with luciferase. In the presence of substrate luciferin and cellular ATP, based on the quantitative production of visible light, the level of transfection will be characterized as effectiveness of virus capture. The quantification of infection will be characterized using a flow cytometer. Cell morphology will be imaged using confocal and optical microscope at 66× and 100× magnification. Bacterial growth can also be monitored in a time-resolved manner using a live-cell microscope system.

As controls, NIPs and different concentrations of non-captured viruses will also be tested for their bacterial infecting ability.

Encapsulation of Titanium Dioxide to Deactivate or Kill the Captured Virus

A virus-degenerating methodology will be incorporated into the virus-imprinted material as mentioned above, through the encapsulation of anti-virus titanium dioxide in the virus-imprinted nanoparticles. Titanium dioxide nanoparticles will be mixed together in the mini-emulsion before polymerization and imprinting, similar to the encapsulation of superparamagnetic nanoparticles. After adsorption of viruses, UV activation of the titanium dioxide will generate free-radicals within the nanoparticles to attack and denature viral capsid proteins, thus killing the viruses.

Titanium dioxide nanoparticles of 5 nm will be purchased and incorporated before the addition of the template during the fabrication procedures. The encapsulated particles (eMIPs) will be characterized by TGA for the amount of titanium dioxide, XPS for surface composition, and SEM and TEM for morphology and size.

As an alternative to titanium dioxide, nano-silver can also be encapsulated as it has been proved to have anti-bacterial effects, although their effects on viruses are less known. The nano-silver (of 5 nm sizes) can be encapsulated in a similar manner to the titanium dioxide.

Assay Of Captured Viruses

The captured viruses from the anti-viral studies will be characterized for their infectivity or bioactivity. The MIPs will be removed from the bacterial culture medium and the viruses desorbed. The desorbed viruses will then be assayed for activity, while the MIPs will be re-used for further virus capture.

### REFERENCES

1) Liu, X.; Guan, Y.; Liu, H.; Ma, Z.; Yang, Y.; Wu, X. J. Magn. Magn. Mater. 2005, 293, 111-118.
2) Shiomi, T.; Matsui, M.; Mizukami, F.; Sakaguchi, K. Biomaterials 2005, 26, 5564-5571.
3) Bonini, F.; Piletsky, S.; Turner, A. P. F.; Speghini, A.; Bossi, A. Biosens. Bioelectron. 2007, 22, 2322-2328.
4) Sambrook and Russel, Molecular Cloning: A Laboratory Manual, Cold Springs Harbor Laboratory, New York (2001)
5) Perez et al., Journal of Applied Polymer Science, Vol. 77, 1851-1859 (2000).
6) Perez-Moral N., Mayes A. G., Analytica Chimica Acta 504 (2004) 15-21.

1. A method of preparing at least one core-shell nanostructure comprising at least one binding site for binding at least one target agent, the method comprising:
(a) providing at least one first hydrophobic polymer to form at least one core;
(b) providing at least one template to bind to the core, wherein the template comprises the conformation of at least one target agent;
(c) providing at least one second hydrophobic polymer to form a shell, the shell contacting at least one portion of the core and at least one portion of the template; and
(d) removing the template to form at least one core-shell nanostructure comprising at least one binding site for binding a target agent.

2. The method according to claim 1, wherein the core-shell nanostructure has a red-blood cell morphology.

3. The method according to claim 1, wherein the binding sites are substantially on the outer face of the shell.

4. The method according to claim 1, wherein the core is magnetic.

5. The method according to claim 1, wherein the first and/-or second hydrophobic polymer is selected from the group consisting of vinyl acrylate polymers, vinyl acetate polymers, acrylamides and/or a mixture thereof.

6. (canceled)

7. The method according to claim 1, wherein the target agent is at least one hydrophilic drug, hydrophobic drug, vitamin, polysaccharide, steroid, cholesterol, protein, DNA, virus, carbohydrate, macrocycle and/or a cell comprising at least one portion of stable conformation.

8. (canceled)

9. (canceled)

10. The method according to claim 1, wherein the method further comprises providing at least one surfactant.

11. The method according to claim 10, wherein the at least one surfactant is selected from the group consisting of polyvinyl alcohol, sodium dodecyl sulfate and cetyl alcohol or a mixture thereof.

12. (canceled)

13. The method according to claim 1, wherein the target agent is bound to the core by covalent bonding in step (b).

14. A core-shell nanostructure obtainable according to the method of claim 1.

15. A core-shell nanostructure comprising:
a hydrophobic polymeric core; and
a hydrophobic polymeric shell on the core,
wherein the shell comprises at least one binding site for binding at least one target agent.

16. The core-shell nanostructure according to claim 15, wherein the core-shell nanostructure has a red-blood cell morphology.

17. The core-shell nanostructure according to claim 15, wherein the binding sites are substantially on the outer face of the shell.

18. The core-shell nanostructure according to claim 15, wherein the core is magnetic.

19. (canceled)

20. (canceled)

21. The core-shell nanostructure according to claim 15, wherein the target agent is at least one hydrophilic drug, hydrophobic drug, vitamin, polysaccharide, steroid, cholesterol protein, DNA, virus, carbohydrate, macrocycle and/or a cell comprising at least one portion of stable conformation.

22. (canceled)

23. (canceled)

24. The core-shell nanostructure according to claim 21, wherein the virus is selected from the group consisting of Retroviruses, Togaviruses, Filoviruses, Herpesviruses, Arenaviruses, Pox viruses, Coronaviruses, Rhobdoviruses, Paramyxoviruses and Orthomyxoviruses.

25. A nanostructure for binding at least one virus, the nanostructure comprising at least one hydrophobic polymer, and at least one binding site on the outer face of the nanostructure for binding the virus.

26. (canceled)

27. (canceled)

28. (canceled)

29. (canceled)

30. A method of detecting and/or imaging at least one target agent in at least one biological sample and/or diagnosis of at least one disorder, the method comprising,
(a) collecting at least one biological sample from a subject;
(b) contacting the nanostructure according to claim 11 to the biological sample;
(c) allowing the nanostructure to contact at least one target agent to faini at least one nanostructure-target agent complex; and
(d) detecting the presence of the nanostructure-target agent complex in the biological sample of the subject, wherein detection of the nanostructure-target agent complex indicates the presence of the target agent and/or the disorder in the subject.

31. (canceled)

32. (canceled)

33. (canceled)

34. A method of treatment of at least one disorder in a subject, the method comprising, administering the nanostructure according to claim 15, to the subject with the disorder.

35. The method of treatment according to claim 34, wherein the disorder is at least one viral infection.

36. (canceled)

37. (canceled)

38. (canceled)

39. (canceled)

40. (canceled)
